(19) United States
(12) Patent Application Publication
(10) Pub. No.: US 2012/0022487 A1
(43) Pub. Date: Jan. 26, 2012

(54) DISPOSABLE URINE ABSORBENT PRODUCT

(76) Inventor: Ikuo Akiyama, Mima-gun (JP)

(21) Appl. No.: 13/260,647

(22) PCT Filed: Nov. 25, 2010
(86) PCT No.: PCT/JP2010/006893

§ 371 Date: Sep. 27, 2011

(30) Foreign Application Priority Data
Dec. 10, 2009 (JP) 2009-280107

(51) Int. Cl. A61F 13/471

(52) U.S. Cl. 604/385.24

(57) ABSTRACT

A disposable urine absorbent product has a sheet-like main body part having an approximately rectangular shape, and an elastic member which lies from one side end of the main body part to the other side end through a front end. In the disposable urine absorbent product, the elastic member which is bonded to the main body part in the state where the elastic member is stretched contracts at left and right sides and a front side of an absorbent core, and therefore, the pair of side ends and the front end in the main body part stand upward at periphery of a front part of the absorbent core and a pocket to house the tip of penis of wearer is formed on the front part of the absorbent core by itself. As above, it is possible to cover the male genitalia with simple structure in the disposable urine absorbent product.

### TECHNICAL FIELD

The present invention relates to a disposable urine absorbent product for men.

### BACKGROUND ART

A product in which the male genitalia of a wearer is housed, is conventionally used as one of absorbent products for receiving excrement from a wearer. For example, in Japanese Utility Model Publication No. 2565071 (Document 1), proposed is formation of a bag-like part in which the male genitalia is housed, where a front part of an incontinence pad is folded in a longitudinal direction so as to face a top sheet, both left and right portions of the front part are folded to the inside and fixed on an adhesive part on the firstly-folded portion.

In Published Japanese Translation No. 5-501977 of PCT International Application No. PCT/SE90/744 (Document 2), proposed is a technique where, by contraction of two elastic members located along two edges of a triangle absorbent pad in a planar view, a portion in the vicinity of an intersection of the two edges is lifted up to cover the male genitalia of a wearer partially. In a pad for a diaper proposed in Japanese Patent Application Laid-Open No. 5-200059 (Document 3), a rectangular absorbent core in a planar view is sandwiched between a rectangular top sheet and a leakage prevention sheet, elastic members each extending in a longitudinal direction are located on both left and right sides of a front part of the absorbent core, portions on which the elastic members are located are folded to the inside and folded back to the outside to be bonded thereto, and therefore a cup-like concave part is formed at the front part.

On the other hand, Japanese Patent Application Laid-Open No. 2008-000480 (Document 4) relates to not an absorbent product for men but a relatively large absorbent product for unisex which is attached at the inside of an underpants or a disposable diaper. In an excrement treating article disclosed in Document 4, an absorbent core where a width of a middle part in a longitudinal direction is less than those of front and back parts (the absorbent core is formed in a form of so-called hourglass) is sandwiched between two rectangular sheets. By contraction of elastic members bonded on flaps surrounding the absorbent core, the flaps all-around the absorbent core are standing toward a wearer, and therefore a concave part to collect excrement is formed.

In each absorbent product of Documents 1 to 3, manufacture of the absorbent product is complicated, because, in order to form the part to house or cover the male genitalia, some portions of the absorbent product are folded and fastened or a shape of the absorbent product is made to be triangular and the elastic members are located along a direction tilted relative to the longitudinal direction of the absorbent product and so on. Also in the absorbent product of Document 1, assembly operation is necessary when wearing the absorbent product, and it puts a burden on the user. On the other hand, in the absorbent product of Document 4, since the elastic members are provided all-around the absorbent core, manufacture of the absorbent product is complicated. If structure of the absorbent product is applied to an absorbent product for men, some elastic members compress the base of the male genitalia and comfort level of the wearer decreases.

### SUMMARY OF INVENTION

The present invention is intended for a disposable urine absorbent product for men. It is an object of the present invention to offer the disposable urine absorbent product to cover the male genitalia with simple structure.

The disposable urine absorbent product according to the present invention comprises:

a rectangular sheet-like main body part; a pair of first elastic members located along left and right side ends of the main body part; and a second elastic member located along a front end of the main body part, wherein the main body part comprises: a liquid-pervious top sheet; a water-repellent or liquid-impervious back sheet; and an absorbent core which is located between the top sheet and the back sheet and which lies at an inner side of the pair of first elastic members and the second elastic member; a width of a front part of the absorbent core, with respect to a left-right direction, is smaller than that of a back part, and the main body part stands upward at periphery of the front part of the absorbent core by contraction of the pair of first elastic members and the second elastic member. In the present invention, it is possible to offer the disposable urine absorbent product to cover the male genitalia with simple structure.

According to a preferred embodiment of the present invention, the second elastic member and the pair of first elastic members are continuous with each other at left and right corners of the front end of the main body part, and are curved at the left and right corners. Therefore, the corners of the front end can be easily made to stand in the disposable urine absorbent product.

According to another preferred embodiment of the present invention, the second elastic member intersects with the pair of first elastic members at left and right corners of the front end of the main body part. Therefore, the corners of the front end can be easily made to stand in the disposable urine absorbent product.

According to still another preferred embodiment of the present invention, since a back end of the main body part has non-elastic property with respect to the left-right direction, comfort level of the wearer can be prevented from decreasing.

According to an aspect of the present invention, a width of a back end of the absorbent core is smaller than the maximum width of the back part of the absorbent core.

According to another aspect of the present invention, the disposable urine absorbent product further comprises a pair of side wall parts located on left and right side portions of the main body part, extending in a front-back direction and standing upward.

These and other objects, features, aspects and advantages of the present invention will become more apparent from the following detailed description of the present invention when taken in conjunction with the accompanying drawings.

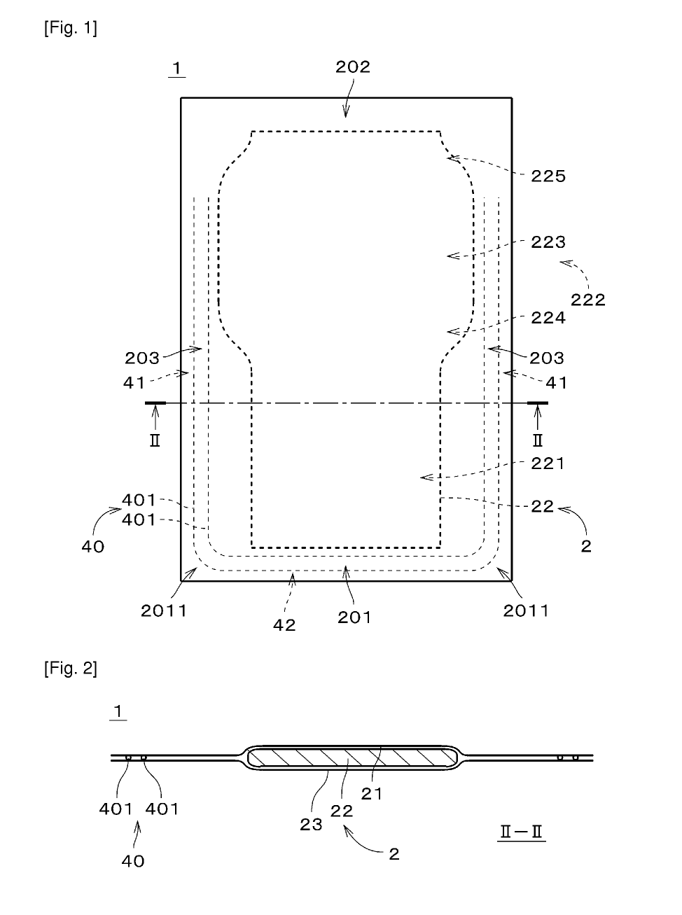

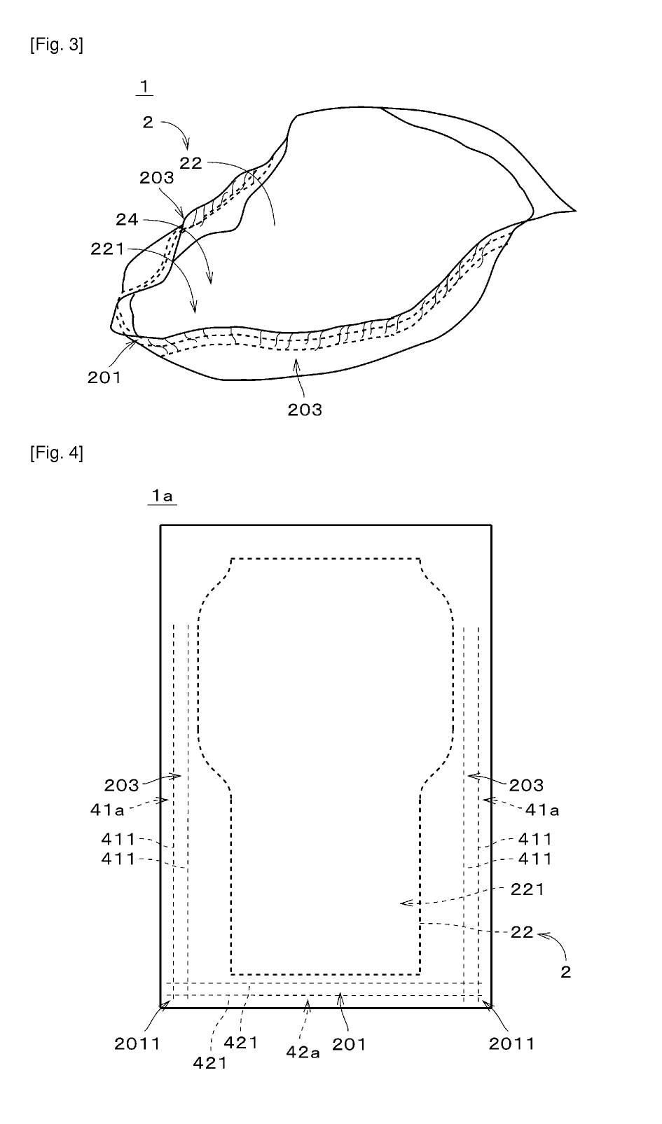

### BRIEF DESCRIPTION OF DRAWINGS

[FIG. 1] FIG. 1 is a plan view of a disposable urine absorbent product in accordance with a first preferred embodiment.

[FIG. 2] FIG. 2 is a cross-sectional view of the disposable urine absorbent product.

[FIG. 3] FIG. 3 is a perspective view of the disposable urine absorbent product.

[FIG. 4] FIG. 4 is a plan view showing another example of disposable urine absorbent

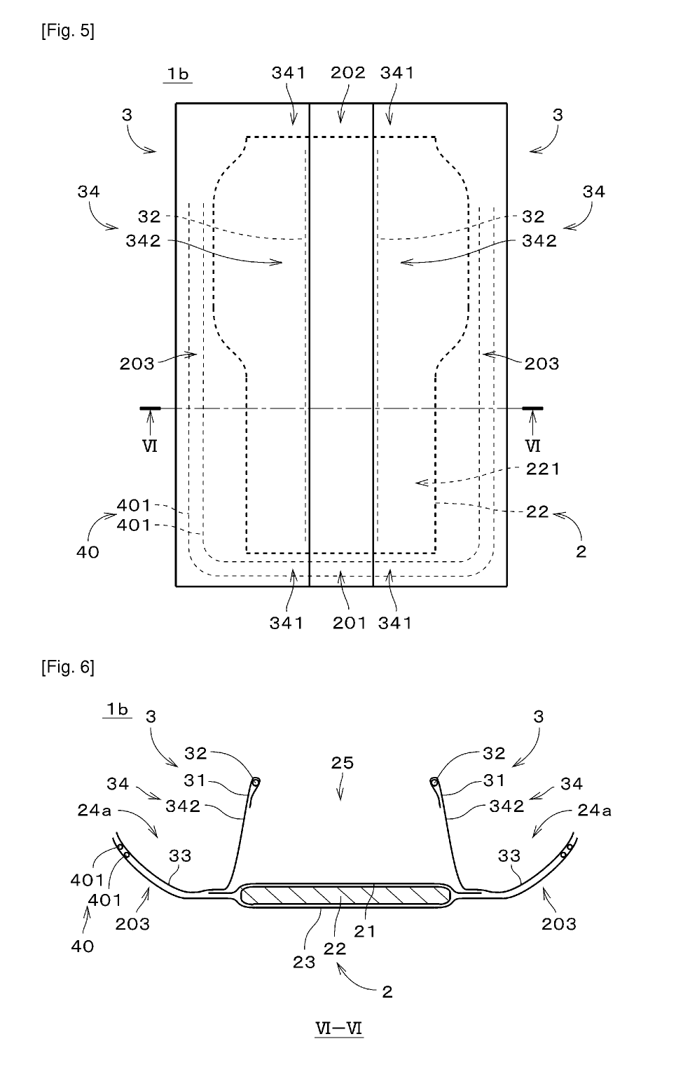

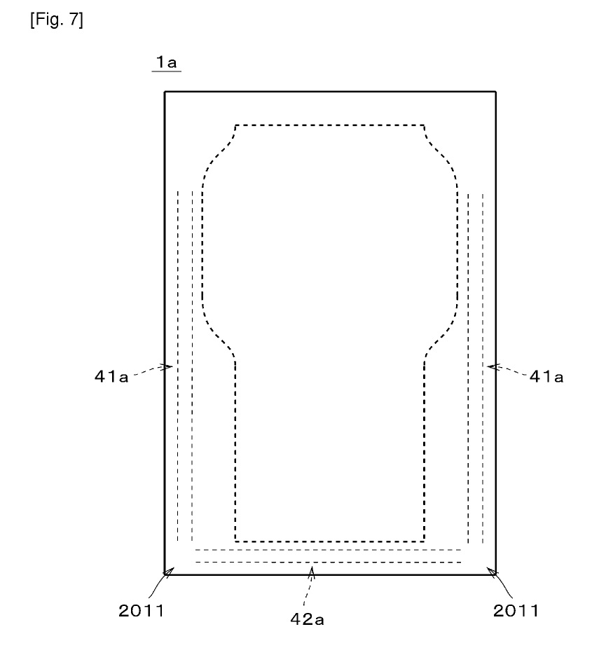

product.

[FIG. 5] FIG. 5 is a plan view of a disposable urine absorbent product in accordance with a second preferred embodiment.

[FIG. 6] FIG. 6 is a cross-sectional view of the disposable urine absorbent product.

[FIG. 7] FIG. 7 is a plan view showing another example of disposable urine absorbent product.

### DESCRIPTION OF EMBODIMENTS

FIG. 1 is a plan view of a disposable urine absorbent product 1 for men in accordance with a first preferred embodiment of the present invention, and FIG. 1 shows a state where the disposable urine absorbent product 1 is developed. The disposable urine absorbent product 1 is an absorbent product attached at the inside of a disposable diaper or the like which is an exterior product worn by a wearer, so as to cover the male genitalia of the wearer, and it receives urine from the wearer. FIG. 1 shows a surface of the disposable urine absorbent product 1, which is to come into contact with the wearer's male genitalia during use, and hereinafter the surface is referred to as an “upper surface”.

As shown in FIG. 1, the disposable urine absorbent product 1 has a sheet-like main body part 2 having an approximately rectangular shape, and an elastic member 40 which lies from one side end 203 of the main body part to the other side end 203 through an end 201 at the lower side in FIG. 1 (the end 201 is a portion corresponding to a tip of the penis of the wearer, and hereinafter referred to as a “front end 201”). The elastic member 40 has two U-shaped elastic yarns 401 which are located so as to surround an after-mentioned absorbent core 22 at both left and right sides and a front side of the absorbent core 22. The two elastic yarns 401 are located along and in almost parallel with each edge of the main body part 2.

Here, in the elastic member 40, each of a pair of portions located along the both left and right side ends 203 is referred to as a first elastic member 41, and a portion located along the front end 201 of the main body part 2 is referred to as a second elastic member 42. In this case, the pair of first elastic members 41 extending in an up-down direction in FIG. 1 (hereinafter, referred to as a “front-back direction”) and the second elastic member 42 extending in a left-right direction orthogonal to the front-back direction are continuous with each other at left and right corners 2011 (i.e., a pair of corners 2011 located at both left and right sides) of the front end 201 of the main body part 2. Portions at which the first elastic members 41 are continuous with the second elastic member 42 are curved at respective corners 2011, and each of them is in the form of convex protruding in a direction away from the absorbent core 22. In other words, the second elastic member 42 and the pair of first elastic members 41 are continuous with each other at the pair of corners 2011 of the main body part 2, and are curved at the corners 2011. In the disposable urine absorbent product 1, no elastic member is provided at a back end 202 of the main body part 2.

FIG. 2 is a cross-sectional view of the disposable urine absorbent product 1 taken along a line II-II in FIG. 1 where the disposable urine absorbent product 1 is cross-sectioned with a plane orthogonal to the front-back direction. As shown in FIG. 2, the main body part 2 has a liquid-pervious top sheet 21, a water-repellent or liquid-impervious back sheet 23, and an absorbent core 22 located between the top sheet 21 and the back sheet 23. The top sheet 21 is a surface to contact with the male genitalia of the wearer. The top sheet 21 and the back sheet 23 are bonded to each other with hot melt adhesive or the like while the elastic member 40 (i.e., the two elastic yarns 401) are located between them at periphery of the absorbent core 22. In FIG. 2, respective constituents of the disposable urine absorbent product 1 are drawn so as to be slightly apart from one another for easy understanding of the drawing.

The top sheet 21 of the main body part 2 is a nonwoven fabric made of liquid-pervious sheet material, for example, hydrophilic fibers, and the top sheet 21 immediately catches moisture of excrement from the wearer and moves the moisture into the absorbent part 22. Examples of nonwoven fabrics used for the top sheet 21 are a point-bond nonwoven fabric, air-through nonwoven fabric, spunlace nonwoven fabric and spunbond nonwoven fabric, and as hydrophilic fibers for making these nonwoven fabrics, normally, cellulose, rayon, cotton and the like are used. As the top sheet 21, a liquid-pervious nonwoven fabric made of hydrophobic fibers (for example, polypropylene, polyethylene, polyester, polyamide or nylon) where hydrophilic treatment is performed on its surface with a surfactant or a porous plastic film may be utilized. In the present embodiment, an air-through nonwoven fabric whose weight per unit area is equal to or more than 10 g/m² and equal to or less than 35 g/m² is used as the top sheet 21.

The absorbent core 22 has a fiber assembly layer which is formed of, for example regenerated hydrophilic fibers (such as cellulose, rayon, cotton), synthetic fibers, pulp fibers or the like. More preferably, the absorbent core 22 further has super absorbent materials which are mixed with the fiber assembly layer and held thereto. Super absorbent polymers (SAP (Super Absorbent Polymer)), super absorbent fibers (SAF (Super Absorbent Fiber)), or the like are used as the super absorbent materials. In the present embodiment, fibrillated pulp fibers are stacked so as to become about 100 to 400 g/m², to form the fiber assembly layer, and granular absorbent resin such as polyacrylic acid, cellulose, starch-acrylonitrile is mixed as the super absorbent materials. Preferably, the absorbent core 22 contains the super absorbent materials where the percentage by weight of the super absorbent materials in the absorbent core 22 is equal to or more than 10 and equal to or less than 70. In the disposable urine absorbent product 1, an absorbent core having no fiber assembly layer (for example, a sheet-like absorbent core where super absorbent materials are fixed between two hydrophilic nonwoven fabric with hot melt adhesive or the like) may be used.

As the back sheet 23, used is a water-repellent or liquid-impervious nonwoven fabric, plastic film, or a laminated sheet of the nonwoven fabric and the plastic film. The back sheet 23 prevents moisture of excrement or the like which has come to the back sheet 23, from leaking out to the outer side of the main body part 2. A spunbond nonwoven fabric, meltblown nonwoven fabric and SMS (spunbond-meltblown-spunbond) nonwoven fabric which are made of hydrophobic fibers, are exemplified as a nonwoven fabric used for the back sheet 23, and water-repellent treatment is performed on the nonwoven fabric as appropriate. In a case where a plastic film is used for the back sheet 23, it is preferable that a plastic film with permeability (breathability) is used, from the viewpoint of preventing sweatiness in the disposable urine absorbent product 1 and providing comfortable feeling to the wearer. In the present embodiment, a plastic film with permeability whose weight per unit area is equal to or more than 10 g/m² and equal to or less than 40 g/m² is used as the back sheet 23.

For example, a polyurethane yarn, strip-like polyurethane film, yarn-like or strip-like natural rubber, or the like is used as the elastic member 40. In the present embodiment, a polyurethane yarn is used as each elastic yarn 401 of the elastic member 40.

As shown in FIG. 1, the absorbent core 22 is located at an inner side of almost U-shaped elastic member 40 (i.e., at an inner side of the pair of first elastic members 41 and the second elastic member 42). In other words, the absorbent core 22 is located away in the left-right direction from the pair of first elastic members 41 at a position between the pair of first elastic members 41 and located away in the front-back direction from the second elastic member 42 in back of the second elastic member 42 (i.e., at a position away from the front end 201 of the main body part 2). The contours of the absorbent core 22 are drawn by thick broken lines in FIG. 1 for easy understanding of the drawing.

The front end 201 of the above main body part 2 is a portion of the main body part 2 extending in the left-right direction in front of the absorbent core 22, and the back end 202 is a portion of the main body part 2 extending in the left-right direction in back of the absorbent core 22. The pair of side ends 203 is portions extending in the front-back direction in left and right of the absorbent core 22. The front end 201, the back end 202 and the pair of side ends 203 in the main body part 2 are formed by bonding the top sheet 21 and the back sheet 23 together.

With respect to the left-right direction, a width of a front part 221 which is a portion of the absorbent core 22 located at the lower side in FIG. 1 (hereinafter, the width in the left-right direction is simply referred to as a “width”) is almost constant. A width of a back part 222 which is a portion of the absorbent core 22 located at the upper side in FIG. 1 is almost constant at a middle portion 223 of the back part 222 in the front-back direction, and the width at a portion 224 in the vicinity of the front part 221 gradually decreases toward the front part 221 (i.e., the width at the portion 224 becomes smaller as it comes close to the front part 221). The width of the back part 222 at a back end 225 of the back part 222 gradually decreases toward the back. In other words, the width of the back end 225 of the absorbent core 22 is smaller than the width of the middle portion 223 which is the maximum width of the back part 222. In the present embodiment, both ends of the elastic member 40 (i.e., back ends of the pair of first elastic members 41) are positioned at left and right sides of a portion in the vicinity of boundary between the middle portion 223 and the back end 225 in the back part 222 of the absorbent core 22.

In the disposable urine absorbent product 1, the elastic member 40 (i.e., the pair of first elastic members 41 and the second elastic member 42) which is bonded to the main body part 2 in the state where the elastic member 40 is stretched (i.e., the stretched elastic member 40 bonded to the main body part 2) contracts at the both left and right sides and the front side of the absorbent core 22 (i.e., in left, right and front of the absorbent core 22), and therefore, as shown in FIG. 3, the pair of side ends 203 and the front end 201 in the main body part 2 stand upward at periphery of the front part 221 of the absorbent core 22 to form a pocket 24 on the front part 221 of the absorbent core 22. When the disposable urine absorbent product 1 is worn, the male genitalia of the wearer is covered with the absorbent core 22, the front end 201 and the side ends 203 where the front end 201 and the side ends 203 are standing at periphery of the absorbent core 22, in the state where the tip of penis of the wearer is housed in the pocket 24.

In the disposable urine absorbent product 1, a shape of the main body part 2 is not a special shape such as a triangle, but a simple shape (almost rectangular shape) as described above. And the disposable urine absorbent product 1 forms the pocket 24 by itself, which is to house the tip of the penis, by contraction of the elastic member 40 without assembly work where some portions of the disposable urine absorbent product are folded and fastened. As above, it is possible to cover the male genitalia with simple structure in the disposable urine absorbent product 1. Therefore, cost for manufacture of the disposable urine absorbent product 1 can be reduced, and the user can use the disposable urine absorbent product 1 easily.

As shown in FIG. 1, in the almost rectangular main body part 2 of the disposable urine absorbent product 1, since the width of the front part 221 of the absorbent core 22 is smaller than that of the back part 222, a width of the side end 203 becomes large at each of the left and right sides of the front part 221 of the absorbent core 22 and the side end 203 stands in tall form by contraction of the elastic member 40. Therefore, the deep pocket 24 (see FIG. 3) is formed and the tip of the penis is housed in the pocket 24 certainly.

In the disposable urine absorbent product 1, the elastic member 40 is curved in the form of convex protruding in the direction away from the absorbent core 22 at each of the left and right corners 2011 of the front end 201 of the main body part 2 (i.e., the second elastic member 42 and the pair of first elastic members 41 are continuous with each other at the pair of corners 2011 of the main body part 2, and are curved at the left and right corners 2011). Consequently, the elastic member 40 is located in the vicinity of edges of the corners 2011, and therefore it is possible to easily make the corners 2011 stand up at the time when the front end 201 and the pair of side ends 203 in the main body part 2 stand up.

Since the width of the back end 225 of the absorbent core 22 is smaller than the maximum width of the back part 222 of the absorbent core 22 in the disposable urine absorbent product 1, the widths of the side ends 203 in the main body part 2 become large at both left and right sides of the back end 225 of the absorbent core 22. Therefore, stiffness of portions at both left and right sides of the back end 202 in the disposable urine absorbent product 1 is decreased, and the portions at the left and right sides of the back end 202 can be easily deformed in accordance with the shape of base of the male genitalia at the time when the male genitalia is covered with the disposable urine absorbent product 1.

As above, since no elastic member is provided at the back end 202 of the main body part 2 in the disposable urine absorbent product 1, the back end 202 has non-elastic property (no stretchability) with respect to the left-right direction and gathers are not formed at the back end 202. Therefore, decrease of comfort level of the wearer by compression of the base of the male genitalia with the gathers can be prevented when the male genitalia is covered with the disposable urine absorbent product 1. In addition, manufacturing process of the disposable urine absorbent product 1 can be simplified as compared to structure where an elastic member is provided all-around the absorbent core 22.

FIG. 4 is a view showing another preferable example of elastic member bonded on the main body part 2. The disposable urine absorbent product la shown in FIG. 4 comprises, as substitute for the elastic member 40 shown in FIG. 1, a pair of first elastic members 41a respectively located along the left and right side ends 203 of the main body part 2, and a second elastic member 42a located along the front end 201 of the main body part 2. The first elastic members 41a have two elastic yarns 411 each extending linearly in almost parallel with the front-back direction, and the second elastic member 42a has two elastic yarns 421 each extending linearly in almost parallel with the left-right direction. The second elastic member 42a is a member separated from the pair of first elastic members 41a, and the second elastic member 42a intersects approximately perpendicularly with the pair of first elastic members 41a at left and right corners 2011 of the front end 201 of the main body part 2.

In the disposable urine absorbent product la, the pair of first elastic members 41a and the second elastic member 42a contract at the left and right sides and the front side of the absorbent core 22, and therefore the pair of side ends 203 and the front end 201 in the main body part 2 stand upward at periphery of the front part 221 of the absorbent core 22 to form a pocket 24 on the front part 221 of the absorbent core 22 in a similar fashion to FIG. 3. The male genitalia of the wearer is covered with the absorbent core 22, the front end 201 and the side ends 203 where the front end 201 and the side ends 203 are standing at periphery of the absorbent core 22, in the state where the tip of penis of the wearer is housed in the pocket 24. As above, it is possible to cover the male genitalia with simple structure in the disposable urine absorbent product la.

In the disposable urine absorbent product la, since the second elastic member 42a intersects with the pair of first elastic members 41a at the pair of corners 2011 in the front end 201 of the main body part 2, the second elastic member 42a and the first elastic members 41a are located in the vicinity of edges of the corners 2011. Therefore, it is possible to easily make the corners 2011 stand up at the time when the front end 201 and the pair of side ends 203 in the main body part 2 stand up. Furthermore, since each of the first elastic members 41a and the second elastic member 42a is linear, manufacturing process of the disposable urine absorbent product can be simplified as compared to the disposable urine absorbent product 1 shown in FIG. 1. On the other hand, in the disposable urine absorbent product 1 shown in FIG. 1, the second elastic member 42 and the pair of first elastic members 41 are continuous with each other at the left and right corners 2011 of the main body part 2, and are curved at the left and right corners 2011. Therefore, when the front end 201 and the side ends 203 are standing, interference between the corners 2011 and portions around the corners 2011 is suppressed, and the front end 201 and the side ends 203 firmly contact with the penis.

Next, discussion will be made on a disposable urine absorbent product in accordance with a second preferred embodiment of the present invention. FIG. 5 is a plan view of a disposable urine absorbent product 1b in accordance with the second preferred embodiment, and FIG. 5 shows a state where the disposable urine absorbent product 1b is developed. FIG. 6 is a cross-sectional view of the disposable urine absorbent product 1b taken along a line VI-VI in FIG. 5 where the disposable urine absorbent product 1b is cross-sectioned with a plane orthogonal to the up-down direction in FIG. 5 (i.e., the front-back direction). As shown in FIGS. 5 and 6, the disposable urine absorbent product 1b comprises the same components as those of the disposable urine absorbent product 1 shown in FIGS. 1 to 3, except for the point that it comprises a pair of side sheets 3 located on both left and right side portions of the main body part 2 and extending in the front-back direction. In the following description, out of components of the disposable urine absorbent product 1b, components corresponding to respective components in the disposable urine absorbent product 1 are denoted by the same reference signs as the respective components. Unlike FIG. 2 described above, FIG. 6 shows a state where portions of after-mentioned side wall part 34 and the side ends 203 of the main body part 2 stand upward (i.e., a state corresponding to FIG. 3 described above).

As shown in FIG. 5, the side sheets 3 are band-like sheet members each extending across almost the entire length of the main body part 2 in the front-back direction. As shown in FIG. 6, a width of the top sheet 21 is smaller than a width of the back sheet 23 in the disposable urine absorbent product 1b, and an outside portion 33 of each side sheet 3 in the left-right direction is bonded on a portion of the top sheet 21 in the vicinity of its side edge in the left-right direction and a portion of the back sheet 23 which is not covered with the top sheet 21, with hot melt adhesive. In the following description, the outside portion 33 of each side sheet 3 is referred to as the “bonded part 33”, and a portion 34 which lies on the inside of the bonded part 33 in the left-right direction and which is continuous with the bonded part 33 is referred to as the “side wall part 34”. Bonding of the side sheet 3 to the top sheet 21, the back sheet 23 and the others may be performed by heat bonding (thermal fusion bonding), ultrasonic bonding or the like.

As shown in FIG. 5, the pair of side wall parts 34 is located on both side portions of the main body part 2, respectively, and extends across almost the entire length thereof in the front-back direction. Preferably, each side wall part 34 is fixed on the main body part 2 with hot melt adhesive (or by heat bonding or ultrasonic bonding) at side wall end parts 341 which are both ends in the front-back direction. In a standing part 342 between the two side wall end parts 341, an elastic member 32 extending in the front-back direction is bonded to the vicinity of an inside edge of the standing part 342 (i.e., the vicinity of a free edge) with hot melt adhesive. By contraction of the elastic member 32, the standing part 342 stands upward as shown in FIG. 6.

The side sheet 3 is formed by folding a sheet main body 31 into two portions at a folding line extending in the front-back direction and by locating (sandwiching in) the elastic member 32 between the two portions overlapped with each other where the sheet main body 31 is a water-repellent or liquid-impervious nonwoven fabric (for example, a spunbond nonwoven fabric, meltblown nonwoven fabric or SMS nonwoven fabric) made of hydrophobic fibers (e.g., polypropylene, polyethylene, polyester, polyamide or nylon). The above two portions of the sheet main body 31 overlapped with each other are bonded to each other by heat bonding or the like.

In the present embodiment, an SMS nonwoven fabric with breathability whose weight per unit area is equal to or more than 10 g/m² and equal to or less than 30 g/m² is used as the sheet main body 31. Water-repellent treatment may be performed on the sheet main body 31 as appropriate. In the disposable urine absorbent product 1b, a plastic film, or a laminated sheet of the above water-repellent or liquid-impervious nonwoven fabric and the plastic film may be used as the sheet main body 31. For example, a polyurethane yarn, strip-like polyurethane film, yarn-like or strip-like natural rubber, or the like is used as the elastic member 32. In the present embodiment, a polyurethane yarn is used as the elastic member 32.

In the disposable urine absorbent product 1b, the elastic yarns 401 of the elastic member 40 are located between the bonded parts 33 of the pair of side sheets 3 and the back sheet 23 at the left and right sides of the absorbent core 22, and are located between the top sheet 21 and the back sheet 23 at the front side of the absorbent core 22. By contraction of the elastic member 40 (i.e., the two elastic yarns 401) which is bonded to the side sheets 3, the top sheet 21 and the back sheet 23 in the state where the elastic member 40 is stretched, the pair of side ends 203 and the front end 201 in the main body part 2 shown in FIG. 5 stand upward at periphery of the front part 221 of the absorbent core 22, and as shown in FIG. 6, a pocket 24a is formed between each side end 203 in the main body part 2 and the standing part 342 in the side sheet 3 adjacent to the side end 203. When the disposable urine absorbent product 1b is worn, the male genitalia is covered with the main body part 2 in the state where the penis is housed in a space 25 between the pair of standing parts 342.

In the disposable urine absorbent product 1b, the pair of side ends 203 and the front end 201 stand up by contraction of the elastic member 40 and the main body part 2 becomes a shape to cover the male genitalia by itself. Therefore, it is possible to cover the male genitalia with simple structure in a similar fashion to the first preferred embodiment. In addition, since the elastic members 32 in the side sheets 3 contract in the front-back direction, the front end 201 and the pair of side ends 203 in the main body part 2 are lifted up to contact with the male genitalia more firmly.

In the disposable urine absorbent product 1b, it can be suppressed that urine leaks from the space 25 to the outsides in the left-right direction, by the pair of standing parts 342. Furthermore, the pockets 24a are formed at the outsides of the pair of standing parts 342 (i.e., the pockets 24a are formed between the pair of standing parts 342 and the pair of side ends 203). Consequently, if urine leaks to the outsides of the space 25, leaked urine can be caught by the pockets 24a, and it is possible to prevent urine from leaking out to the outer side of the disposable urine absorbent product 1b.

Though the preferred embodiments of the present invention have been discussed above, the present invention is not limited to the above-discussed preferred embodiments, but allows various variations.

In the disposable urine absorbent product 1 shown in FIG. 1, the elastic member 40 is not necessarily curved at the corners 2011 of the main body part 2. For example, the elastic member 40 may extend almost linearly in a direction tilted relative to both the front-back direction and the left-right direction at the corners 2011. In the disposable urine absorbent product 1b shown in FIG. 4, the second elastic member 42a does not necessarily intersect with the pair of first elastic members 41a at the pair of corners 2011. For example, as shown in FIG. 7, a length of the second elastic member 42a in the left-right direction is made to be slightly shorter than a distance between the pair of first elastic members 41a in the left-right direction, and the corners 2011 may be non-intersections between the first elastic members 41a and the second elastic member 42a.

In the disposable urine absorbent products in accordance with the above preferred embodiments, as long as decrease of comfort level of the wearer is within allowed limits, elastic property may be provided to some portion of the back end 202 in the main body part 2. In the back part 222 of the absorbent core 22, as long as the main body part 2 can be easily deformed along the male genitalia, the width of the back end 225 may be equivalent to the width of the middle portion 223.

While the invention has been shown and described in detail, the foregoing description is in all aspects illustrative and not restrictive. It is therefore understood that numerous modifications and variations can be devised without departing from the scope of the invention.

### REFERENCE SIGNS LIST

1, 1a, 1b disposable urine absorbent product
2 main body part
21 top sheet
22 absorbent core
23 back sheet
34 side wall part
40 elastic member
41, 41a first elastic member
42, 42a second elastic member
201 front end
202 back end
203 side end
221 front part
222 back part
225 back end
2011 corner

1-6. (canceled)

7. A disposable urine absorbent product for men, comprising:
a rectangular sheet-like main body part;
a pair of first elastic members located along left and right side ends of said main body part; and
a second elastic member located along a front end of said main body part, wherein said main body part comprises:
a liquid-pervious top sheet;
a water-repellent or liquid-impervious back sheet; and
an absorbent core which is located between said top sheet and said back sheet and which lies at an inner side of said pair of first elastic members and said second elastic member;
a width of a front part of said absorbent core, with respect to a left-right direction, is smaller than that of a back part, and
said main body part stands upward at periphery of said front part of said absorbent core by contraction of said pair of first elastic members and said second elastic member.

8. The disposable urine absorbent product according to claim 7, wherein said second elastic member and said pair of first elastic members are continuous with each other at left and right corners of said front end of said main body part, and are curved at said left and right corners.

9. The disposable urine absorbent product according to claim 7, wherein
said second elastic member intersects with said pair of first elastic members at left and right corners of said front end of said main body part.

10. The disposable urine absorbent product according to claim 7, wherein
a back end of said main body part has non-elastic property with respect to said left-right direction.

11. The disposable urine absorbent product according to claim 7, wherein
a width of a back end of said absorbent core is smaller than the maximum width of said back part of said absorbent core.

12. The disposable urine absorbent product according to claim 7, further comprising
a pair of side wall parts located on left and right side portions of said main body part, extending in a front-back direction and standing upward.

13. The disposable urine absorbent product according to claim 8, further comprising
a pair of side wall parts located on left and right side portions of said main body part, extending in a front-back direction and standing upward.

14. The disposable urine absorbent product according to claim 9, further comprising
a pair of side wall parts located on left and right side portions of said main body part, extending in a front-back direction and standing upward.

15. The disposable urine absorbent product according to claim 10, further comprising
a pair of side wall parts located on left and right side portions of said main body part, extending in a front-back direction and standing upward.

16. The disposable urine absorbent product according to claim 11, further comprising
a pair of side wall parts located on left and right side portions of said main body part, extending in a front-back direction and standing upward.
